(19) United States
(12) Patent Application Publication
(10) Pub. No.: US 2026/0251649 A1
(43) Pub. Date: Aug. 27, 2026

(54) INTEGRATED DEVICE FOR SINGLE-STEP EXTRACTION AND AFFINITY CAPTURE OF ANALYTES FROM DRIED BIOLOGICAL SAMPLES

(71) Applicant: QuantiScientifics, LLC, Orange, CA (US)

(72) Inventor: Robert Stanley Matson, Orange, CA (US)

(73) Assignee: QuantiScientifics, LLC, Orange, CA (US)

(21) Appl. No.: 19/545,806

(22) Filed: Feb. 20, 2026

Related U.S. Application Data
(60) Provisional application No. 63/764,392, filed on Feb. 27, 2025.

(51) Int. Cl. G01N 33/543; B01L 3/00; G01N 21/64; G01N 33/68

(52) U.S. Cl. CPC G01N 33/54386; B01L 3/502715; G01N 21/64; G01N 33/6803; B01L 2200/16; B01L 2300/0816; G01N 2333/91245; G01N 2800/00

(57) ABSTRACT

An integrated device and method are disclosed for single-step extraction and affinity capture of analytes from dried biological sample carriers. The device defines an internal cavity configured to fully receive a sample carrier comprising a dried biological sample, such as a dried blood spot. Multiple analyte-specific capture elements are immobilized on a surface within the cavity. Upon introduction of an extraction buffer, analytes are released from the dried sample and concurrently captured by the immobilized capture elements within the same device, eliminating intermediate elution, transfer, or washing steps. Continuous depletion of free analyte within the capture zone promotes further analyte release from the dried sample, enabling efficient equilibrium-driven extraction. The device is compatible with immunoassay and other affinity-based detection formats and supports proteins, nucleic acids, and small-molecule analytes. The disclosed device reduces processing time, handling complexity, and sample loss, and is suitable for diagnostic, screening, and monitoring applications.

### CROSS-REFERENCE TO RELATED APPLICATIONS

This application claims priority from prior utility provisional application with the application No. 63/764,392 filed on Feb. 27, 2025. The entire collective teachings thereof being herein incorporated by reference.

### STATEMENT REGARDING FEDERALLY SPONSORED RESEARCH OR DEVELOPMENT

Not Applicable.

### REFERENCE TO A SEQUENCE LISTING, A LARGE TABLE, OR A COMPUTER PROGRAM LISTING APPENDIX ON READ-ONLY OPTICAL DISC (IF APPLICABLE)

Not Applicable.

### FIRST NAMED INVENTOR

Robert Stanley Matson.

### FIELD OF THE INVENTION

The present invention relates generally to diagnostic and analytical testing of biological samples. More particularly, the invention pertains to an integrated device and method for single-step extraction and affinity capture of analytes from dried biological sample carriers, including dried blood spot (DBS) samples.

### BACKGROUND OF THE INVENTION

The use of dried blood spot (DBS) paper carriers as blood sample receptacles in diagnostic testing is well established and widely practiced worldwide. DBS sampling was originally introduced in 1961 by Robert Guthrie for newborn screening of phenylketonuria (PKU). In this approach, a small volume of blood obtained via heel prick is spotted onto filter paper and allowed to dry, enabling simplified sample collection, safe storage, and cost-effective transport. DBS methodologies were subsequently adopted for the assessment of thyroxine (T4) and thyroid-stimulating hormone (TSH) in congenital hypothyroidism, as well as for newborn screening of cystic fibrosis.

DBS-based sampling has also been explored for serological and infectious disease testing. Early studies demonstrated the feasibility of transporting infectious blood samples on DBS substrates for antibody detection, including screening for human African trypanosomiasis. Subsequent investigations evaluated recovery of immunoglobulins from DBS in comparison to serum and plasma samples, demonstrating general concordance but significantly reduced absolute analyte recovery from DBS. For example, immunoglobulin G (IgG) concentrations recovered from DBS were reported to be substantially lower than those from serum or plasma, necessitating large correction factors and prolonged elution times to achieve acceptable correlation.

Despite its logistical advantages, analyte extraction from DBS samples is conventionally performed using multi-step, time-intensive workflows. Typical protocols require a DBS punch to be placed into a dedicated elution plate containing extraction buffer and incubated for extended periods, often overnight (18-20 hours) at low temperature. Following extraction, the eluate must be transferred to a separate assay plate for immunoassay development. Such workflows increase assay time, handling complexity, risk of sample loss or contamination, and variability introduced by manual transfer steps. Standardized protocols, including those disseminated by the Centers for Disease Control & Prevention, exemplify this two-step elution-and-transfer paradigm.

More recent approaches have attempted to reduce processing time or simplify handling, including DBS strip formats that eliminate punching. However, these systems continue to rely on sequential extraction and assay steps, often requiring multiple hours of extraction followed by separate assay development. As a result, existing DBS-based diagnostic systems remain poorly suited for rapid testing, high-throughput screening, or point-of-care applications where speed, simplicity, and minimal handling are critical.

Accordingly, there remains a clear and unmet need for a device and method that enable rapid and efficient analyte extraction from DBS samples while eliminating intermediate transfer steps and reducing overall assay time.

### BRIEF SUMMARY OF THE INVENTION

The first aspect of the present invention provides a device for single-step extraction and capture of an analyte from a dried biological sample. The device includes a sensing unit defining at least one internal cavity. The device includes multiple surface-immobilized analyte-specific capture elements. The capture elements are immobilized on a surface within the sensing unit. A sample carrier comprising a dried biological sample is dimensioned to be fully received within the internal cavity of the sensing unit. The dried biological sample comprises one or more analytes. An extraction buffer introduced into the internal cavity is configured to release the analytes from the sample carrier while enabling direct binding of the released analytes to the multiple surface-immobilized analyte-specific capture elements within the sensing unit.

In an embodiment, the sample carrier comprises a dried blood spot (DBS) punch derived from a Guthrie card.

In another embodiment, the sensing unit comprises a microwell, microchamber, microfluidic well, or reaction cartridge.

In yet another embodiment, the analyte-specific capture elements comprise antibodies, nanobodies, aptamers, nucleic acids, enzymes, phage-derived binders, inorganic cages, or combinations thereof.

In yet another embodiment, the analytes comprise proteins, nucleic acids, lipids, carbohydrates, sugars, drug molecules, cells, viruses, organelle-derived substances, membrane-derived substances, or a modification or derivative thereof.

In yet another embodiment, the extraction buffer is formulated to release the analytes from the dried biological sample without substantially inhibiting affinity binding between the analytes and the surface-immobilized analyte-specific capture elements.

In yet another embodiment, the dried biological sample is derived from whole blood, serum, plasma, saliva, urine, bile, vaginal fluid, tear fluid, sweat, or cerebrospinal fluid.

In yet another embodiment, the dried biological sample is derived from skin, hair, nasal aspirates, or fecal material.

In yet another embodiment, the analyte capture occurs concurrently with analyte extraction within the sensing unit without transferring the sample carrier or released analytes to a separate extraction or capture vessel.

In yet another embodiment, detection of the captured analyte is performed using colorimetric, fluorescent, or luminescent signal detection.

In yet another embodiment, detection of the captured analyte is performed using a mass-sensing technique or a signal amplification process. When the captured analyte comprises a nucleic acid, detection is carried out by polymerase chain reaction (PCR) or a CRISPR-based detection process. When the captured analyte comprises a protein, detection is carried out by catalytic signal amplification, an energy transfer-based detection process, or a CRISPR-mediated detection process. The captured analyte, whether a nucleic acid or a protein, is optionally associated with or attached to a component selected from a protein, nucleic acid, lipid, carbohydrate, sugar, drug molecule, cell, virus, organelle-derived substance, membrane-derived substance, or a modification or derivative thereof.

The second aspect of the present invention provides a method for single-step extraction and capture of an analyte from a dried biological sample. The method includes (i) placing a sample carrier comprising a dried biological sample containing one or more analytes into an internal cavity of a sensing unit having multiple surface-immobilized analyte-specific capture elements; (ii) introducing an extraction buffer into the sensing unit to release the analytes from the sample carrier; and (iii) capturing the released analytes directly on the multiple surface-immobilized analyte-specific capture elements within the sensing unit.

In an embodiment, the analyte capture occurs concurrently with analyte extraction within the sensing unit without transferring the sample carrier or released analytes to a separate extraction or capture vessel.

In another embodiment, the sample carrier comprises a dried blood spot (DBS) punch derived from a Guthrie card and the sensing unit comprises a microwell, microchamber, microfluidic well, or reaction cartridge.

In yet another embodiment, the analyte-specific capture elements comprise antibodies, nanobodies, aptamers, nucleic acids, enzymes, phage-derived binders, inorganic cages, or combinations thereof.

In yet another embodiment, the analytes comprise proteins, nucleic acids, lipids, carbohydrates, sugars, drug molecules, cells, viruses, organelle-derived substances, membrane-derived substances, or a modification or derivative thereof.

The present invention offers significant advantages over conventional systems for processing dried biological samples. The device is configured to fully enclose the sample carrier during analyte extraction and capture, thereby reducing sample handling, minimizing contamination risk, and preventing analyte loss associated with transfer between separate vessels. By eliminating intermediate transfer, elution, or washing steps between extraction and capture, the disclosed system simplifies workflow, shortens assay time, and improves reproducibility by reducing operator-dependent variability. Concurrent extraction and affinity capture within the same device enables continuous depletion of free analyte in solution, which promotes enhanced analyte release from the dried sample and improves extraction efficiency. The integrated design supports use in diagnostic, screening, and monitoring assays and is compatible with a broad range of analytes and detection formats. Additionally, the system may be implemented in both point-of-care and laboratory-based settings, enabling flexible deployment for rapid testing, decentralized diagnostics, and high-throughput analytical applications.

The embodiments covered by this patent are defined by the claims. The summary above provides a general overview of various aspects and introduces some of the concepts that are discussed in greater detail in the following description section. This summary is not meant to identify the key or essential features of the claimed subject matter, nor is it intended to be used on its own to determine the scope of the claims. The subject matter should be understood with reference to the entire specification, including any relevant drawings and the claims themselves.

### BRIEF DESCRIPTION OF THE DRAWINGS

A clear understanding of the key features of the invention summarized above may be had by reference to the appended drawings, which illustrate the method and system of the invention, although it will be understood that such drawings depict preferred embodiments of the invention and, therefore, are not to be considered as limiting its scope with regard to other embodiments which the invention is capable of contemplating. Accordingly:

FIG. 1 illustrates an integrated device for single-step extraction and affinity capture of analytes from dried biological samples according to various embodiments of the present invention.

FIG. 2 illustrates an example embodiment of a one-step dried blood spot (DBS) extraction and analyte capture process for assessment of allergenic response using an immunoassay, as implemented within the system illustrated in FIG. 1, according to various embodiments of the present invention.

FIGS. 3A-F, with reference to FIG. 1, illustrates a device-level model explaining how concurrent affinity capture within the device actively promotes continued extraction of analyte from the dried blood spot (DBS) punch over time, according to various embodiments of the present invention.

FIG. 4 illustrates an experimentally derived correlation between specific IgG4 concentrations measured from dried blood spot (DBS) samples and corresponding plasma samples obtained from the same patient according to various embodiments of the present invention.

FIG. 5 is a flow chart illustrating a method for single-step extraction and capture of an analyte from a dried biological sample according to various embodiments of the present invention.

Like reference numerals refer to like parts throughout the several views of the drawings.

### DETAILED DESCRIPTION OF THE INVENTION

The terminology used herein is for the purpose of describing particular embodiments only and is not intended to be limiting of the invention. As used herein, the term “and/or” includes any and all combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

Unless otherwise defined, all terms (including technical and scientific terms) used herein have the same meaning as commonly understood by one having ordinary skill in the art to which this invention belongs. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

In describing the invention, it will be understood that a number of techniques and steps are disclosed. Each of these has individual benefit and each can also be used in conjunction with one or more, or in some cases all, of the other disclosed techniques. Accordingly, for the sake of clarity, this description will refrain from repeating every possible combination of the individual steps in an unnecessary fashion.

Nevertheless, the specification and claims should be read with the understanding that such combinations are entirely within the scope of the invention and the claims. An integrated device for single-step extraction and affinity capture of analytes from dried biological samples is discussed herein. In the following description, for purposes of explanation, numerous specific details are set forth in order to provide a thorough understanding of the present invention. It will be evident, however, to one skilled in the art that the present invention may be practiced without these specific details. The present disclosure is to be considered as an exemplification of the invention and is not intended to limit the invention to the specific embodiments illustrated by the figures or description below. The present invention will now be described by referencing the appended figures representing preferred embodiments.

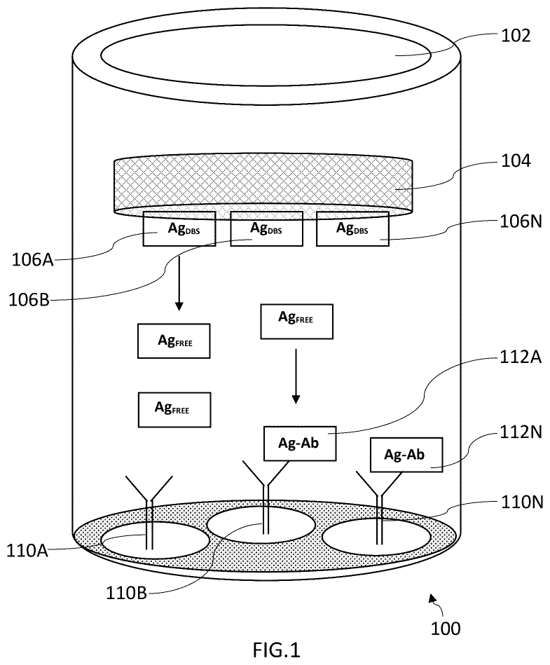

FIG. 1 illustrates an integrated device for single-step extraction and affinity capture of analytes from dried biological samples according to various embodiments of the present invention. The device 100 includes a sensing unit 102 in the form of a microplate well defining an internal cavity. The internal surface of the sensing unit 102, preferably a bottom surface, is functionalized with surface-immobilized analyte-specific capture elements 110A-N, such as antibodies, thereby forming an immune-sensing interface. The device 100 includes a sample carrier 104, exemplified as a DBS punch obtained from a Guthrie card and containing an adsorbed biological sample 106A-N is dimensioned to be fully received and enclosed within the internal cavity of the sensing unit 102. The adsorbed biological sample 106A-N includes analytes associated with a dried biological sample matrix of the sample carrier 104, shown for example as antigen in a DBS punch. An extraction buffer is introduced into the internal cavity such that the sample carrier 104 is fully immersed. The extraction buffer facilitates desorption of the analytes 106A-106N from the sample carrier 104, resulting in hydration and solubilization of the analytes. The released analytes freely diffuse within the internal cavity toward the surface-immobilized analyte-specific capture elements 110A-N. The analytes 106A-106N specifically binds to the surface-immobilized analyte-specific capture elements 110A-N to form an analyte-capture complex 112A-N on the sensing surface. The device 100 enables integrated extraction and capture of the analyte within a single enclosed structure, eliminating the need for intermediate transfer, elution, or separate capture vessels. The configuration supports downstream detection of the captured analyte using colorimetric, fluorescent, or luminescent signal detection modalities.

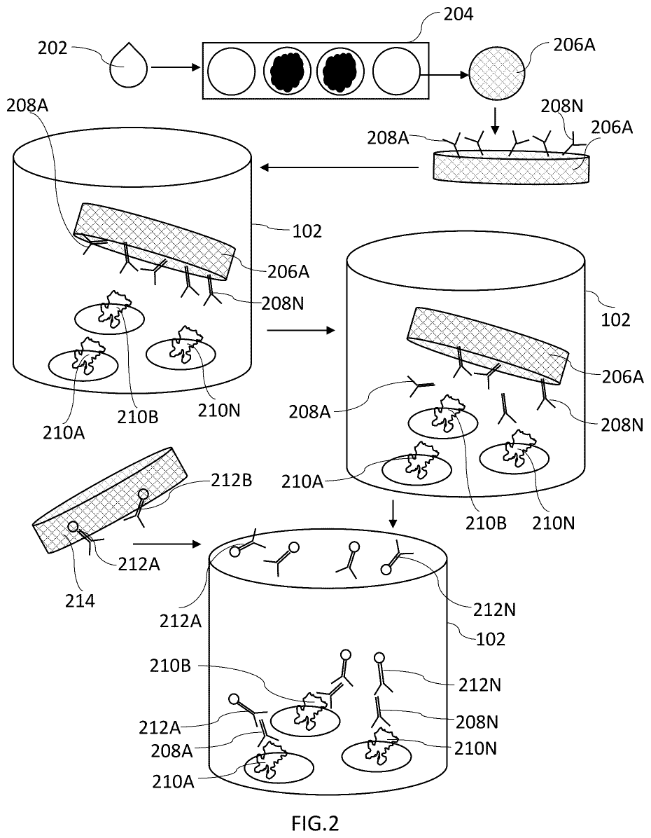

FIG. 2 illustrates an example embodiment of a one-step dried blood spot (DBS) extraction and analyte capture process for assessment of allergenic response using an immunoassay, as implemented within the system illustrated in FIG. 1, according to various embodiments of the present invention. As shown in FIG. 2, blood 202 is first collected from a subject and deposited onto a DBS card 204, where the blood droplets are allowed to dry, resulting in adsorbed biological samples on the card 204. After drying, one or more punches 206A-N are obtained from the DBS card 204 for extraction of sample analytes. In this embodiment, the analytes 208A-N include allergen-responsive immunoglobulins (IgX), such as IgE, IgG, IgA, and IgM. The DBS punch 206A is then transferred into the sensing unit 102 and immersed in the extraction buffer. During an incubation period of approximately one to two hours, the immunoglobulin analytes 208A-N, along with other proteins and biomolecules, are released from the DBS punch 206A into the solution phase and diffuse toward the bottom of the sensing unit 102. The bottom surface of the sensing unit 102 contains a microarray of immobilized protein allergens 210A-N that function as the immunosensor. Immunoglobulins having binding specificity to particular allergens bind to their corresponding immobilized allergens, thereby being captured and removed from solution. Binding of specific immunoglobulins facilitates further release and capture of additional analytes from the DBS punch 206A. Once binding saturation is achieved, the sensing unit 102 may be rinsed and prepared for subsequent development and detection steps of the immunoassay. Following capture of the immunoglobulin analytes 208A-N on the microarray of immobilized protein allergens 210A-N, signal reporter secondary antibodies 212A-N are delivered into the sensing unit 102 via a punch 214. The signal reporter secondary antibodies 212A-N are configured to bind selectively to the captured immunoglobulin analytes 208A-N and includes a detectable reporter moiety. Upon incubation, the signal reporter secondary antibodies 212A-N binds to the captured immunoglobulin analytes 208A-N, thereby enabling development of a measurable signal during subsequent immunoassay processing.

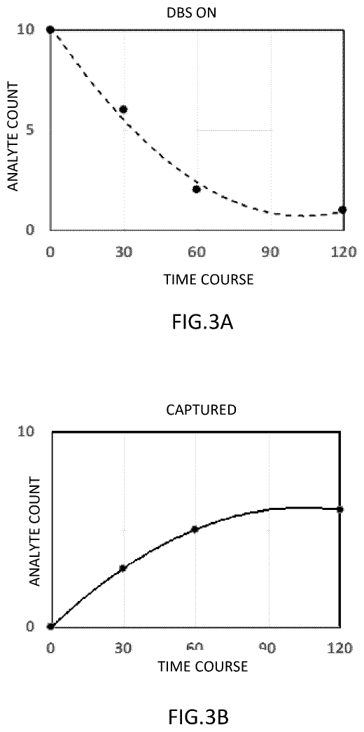

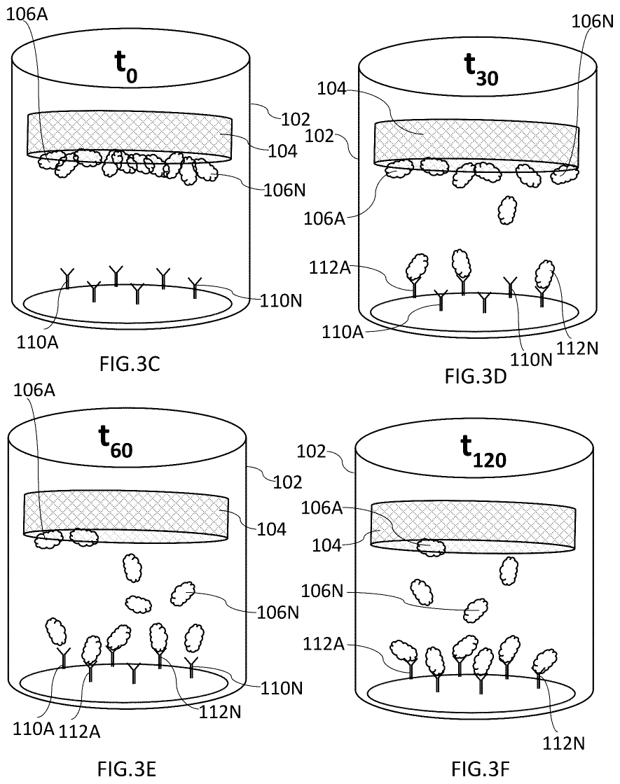

FIGS. 3A-F, with reference to FIG. 1, illustrates a device-level model explaining how concurrent affinity capture within the device actively promotes continued extraction of analyte from the dried blood spot (DBS) punch over time, according to various embodiments of the present invention. Plots 3A-B depict analyte distribution as a function of time. The plot 3A (“DBS on”) represents analytes 106A-N remaining associated with the DBS punch 104, while the plot 3B (“Captured”) represents analytes 106A-N captured on the surface-immobilized analyte-specific capture elements 110A-N within the sensing unit 102. As the process progresses, analyte content on the DBS punch 104 decreases while captured analyte correspondingly increases.

FIGS. 3C-3F illustrate sequential stages of operation within the device 100, wherein a dried blood spot (DBS) punch 104 containing the retained analytes 106A-N is positioned above a sensing surface comprising a microarray of the surface-immobilized analyte-specific capture elements 110A-N including immobilized cognate capture antibodies. Upon introduction of the extraction buffer into the sensing unit 102, analytes 106A-N are released from the DBS punch 104 into solution and diffuses toward the capture surface, where the released analytes 106A-N selectively binds to corresponding antibodies within a capture zone to form immobilized analyte-antibody complexes 112A-N. As analyte is sequestered on the capture surface, the concentration of free analyte in solution is locally depleted, thereby maintaining a concentration gradient between the DBS punch 104 and the capture zone that promotes continued desorption and release of additional analyte from the DBS punch 104 in accordance with equilibrium-driven mass transfer behavior.

At t₀ (FIG. 3C), analytes 106A-N remains predominantly retained within the dried biological sample matrix of the DBS punch 104, with minimal analytes 106A-N present in solution and the surface-immobilized analyte-specific capture elements 110A-N being largely unoccupied. At t₃₀ (FIG. 3D), initial desorption of analytes 106A-N occurs upon hydration by the extraction buffer, and released analytes 106A-N begins diffusing toward and binding to the surface-immobilized analyte-specific capture elements 110A-N, thereby establishing an analyte concentration gradient. At t₆₀ (FIG. 3E), continued sequestration of analytes 106A-N on the capture surface further depletes free analytes 106A-N from solution, sustaining the concentration gradient and promoting additional analyte release from the DBS punch 104. At t₁₂₀ (FIG. 3F), the coupled extraction-capture process approaches a limiting condition, wherein available antibody binding sites of the surface-immobilized analyte-specific capture elements 110A-N may be substantially occupied or analyte levels become stoichiometrically constrained, resulting in reduced or substantially arrested further analyte release and capture. FIGS. 3A-3F demonstrate how the device architecture and immobilized capture chemistry cooperate to enhance analyte extraction from a dried biological sample through continuous depletion of free analyte within the capture zone, without requiring intermediate transfer, washing, or separate extraction steps.

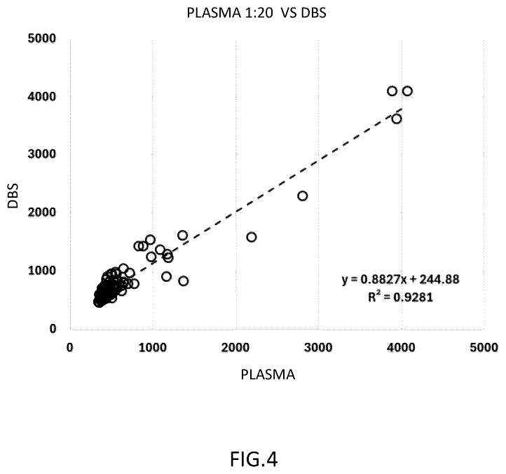

FIG. 4 illustrates an experimentally derived correlation between specific IgG4 concentrations measured from dried blood spot (DBS) samples and corresponding plasma samples obtained from the same patient according to various embodiments of the present invention. The graph plots IgG4 concentration values (ng/mL) determined from DBS-based measurements against IgG4 concentration values obtained from plasma-based measurements, thereby assessing agreement and quantitative comparability between the two sample formats. In the experiment, a microplate well microarray comprising 96 immobilized food protein extracts was incubated for approximately two hours at room temperature with gentle shaking in the presence of an extraction buffer (100 μL). Each well contained either a single 6 mm diameter DBS punch or a plasma sample diluted 1:20 (v/v). Following incubation, the wells were rinsed with a wash buffer, and a fluorescent immunoassay (FIA) was performed using a biotinylated anti-human IgG4 detection antibody, followed by a streptavidin-fluorescent dye conjugate. Fluorescence signal intensities were converted to relative IgG4 concentrations (ng/mL) using calibration curves generated from human IgG4 standards and analyzed by four-parameter logistic regression (4PL). The resulting plot demonstrates a strong positive correlation between IgG4 values obtained from DBS and plasma samples, indicating that DBS-based single-step extraction and capture provides quantitative IgG4 measurements comparable to those derived from conventional plasma samples.

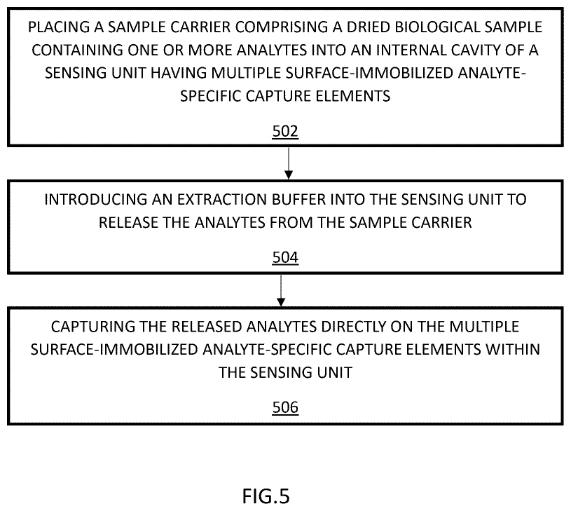

FIG. 5 is a flow chart illustrating a method for single-step extraction and capture of an analyte from a dried biological sample according to various embodiments of the present invention. The method at step 502, includes placing a sample carrier comprising a dried biological sample containing one or more analytes into an internal cavity of a sensing unit having multiple surface-immobilized analyte-specific capture elements. The method at step 504, includes introducing an extraction buffer into the sensing unit to release the analytes from the sample carrier. The method at step 506, includes capturing the released analytes directly on the multiple surface-immobilized analyte-specific capture elements within the sensing unit.

While the present invention has been described in terms of particular embodiments and applications, in both summarized and detailed forms, it is not intended that these descriptions in any way limit its scope to any such embodiments and applications. It will be understood that many substitutions, changes and variations in the described embodiments, applications and details of the method and system illustrated herein and of their operation can be made by those skilled in the art without departing from the spirit of this invention.

What is claimed is:

1. A device for single-step extraction and capture of an analyte from a dried biological sample, wherein the device comprises,
a sensing unit defining at least one internal cavity;
multiple surface-immobilized analyte-specific capture elements, wherein the capture elements are immobilized on a surface within the sensing unit;
a sample carrier comprising a dried biological sample, wherein the dried biological sample comprises one or more analytes, wherein the sample carrier is dimensioned to be fully received within the internal cavity of the sensing unit; and
an extraction buffer configured to release the analytes from the sample carrier while enabling direct binding of the released analytes to the multiple surface-immobilized analyte-specific capture elements within the sensing unit.

2. The device of claim 1, wherein the sample carrier comprises a dried blood spot (DBS) punch derived from a Guthrie card.

3. The device of claim 1, wherein the sensing unit comprises a microwell, microchamber, microfluidic well, or reaction cartridge.

4. The device of claim 1, wherein the analyte-specific capture elements comprise antibodies, nanobodies, aptamers, nucleic acids, enzymes, phage-derived binders, inorganic cages, or combinations thereof.

5. The device of claim 1, wherein the analytes comprise proteins, nucleic acids, lipids, carbohydrates, sugars, drug molecules, cells, viruses, organelle-derived substances, membrane-derived substances, or a modification or derivative thereof.

6. The device of claim 1, wherein the extraction buffer is formulated to release the analytes from the dried biological sample without substantially inhibiting affinity binding between the analytes and the surface-immobilized analyte-specific capture elements.

7. The device of claim 1, wherein the dried biological sample is derived from whole blood, serum, plasma, saliva, urine, bile, vaginal fluid, tear fluid, sweat, or cerebrospinal fluid.

8. The device of claim 1, wherein the dried biological sample is derived from skin, hair, nasal aspirates, or fecal material.

9. The device of claim 1, wherein the analyte capture occurs concurrently with analyte extraction within the sensing unit without transferring the sample carrier or released analytes to a separate extraction or capture vessel.

10. The device of claim 1, wherein detection of the captured analyte is performed using colorimetric, fluorescent, or luminescent signal detection.

11. The device of claim 1, wherein detection of the captured analyte is performed using a mass-sensing technique or a signal amplification process,
wherein, when the captured analyte comprises a nucleic acid, detection is carried out by polymerase chain reaction (PCR) or a CRISPR-based detection process,
wherein, when the captured analyte comprises a protein, detection is carried out by catalytic signal amplification, an energy transfer-based detection process, or a CRISPR-mediated detection process, and
wherein the captured analyte, whether a nucleic acid or a protein, is optionally associated with or attached to a component selected from a protein, nucleic acid, lipid, carbohydrate, sugar, drug molecule, cell, virus, organelle-derived substance, membrane-derived substance, or a modification or derivative thereof.

12. A method for single-step extraction and capture of an analyte from a dried biological sample, comprising:
placing a sample carrier comprising a dried biological sample containing one or more analytes into an internal cavity of a sensing unit having multiple surface-immobilized analyte-specific capture elements;
introducing an extraction buffer into the sensing unit to release the analytes from the sample carrier; and
capturing the released analytes directly on the multiple surface-immobilized analyte-specific capture elements within the sensing unit.

13. The method of claim 12, wherein the analyte capture occurs concurrently with analyte extraction within the sensing unit without transferring the sample carrier or released analytes to a separate extraction or capture vessel.

14. The method of claim 12, wherein the sample carrier comprises a dried blood spot (DBS) punch derived from a Guthrie card, wherein the sensing unit comprises a microwell, microchamber, microfluidic well, or reaction cartridge.

15. The method of claim 12, wherein the analyte-specific capture elements comprise antibodies, nanobodies, aptamers, nucleic acids, enzymes, phage-derived binders, inorganic cages, or combinations thereof.

16. The method of claim 12, wherein the analytes comprise proteins, nucleic acids, lipids, carbohydrates, sugars, drug molecules, cells, viruses, organelle-derived substances, membrane-derived substances, or a modification or derivative thereof.
